(19) United States
(12) Patent Application Publication
(10) Pub. No.: US 2025/0073274 A1
(43) Pub. Date: Mar. 6, 2025

(54) METHODS FOR TREATING CD33-POSITIVE HEMATOLOGICAL MALIGNANCIES

(71) Applicant: Actinium Pharmaceuticals, Inc., New York, NY (US)

(72) Inventors: Dale L. Ludwig, New York, NY (US); Sandesh Seth, New York, NY (US)

(21) Appl. No.: 18/728,076

(22) PCT Filed: Jan. 11, 2023
(86) PCT No.: PCT/US2023/060492

§ 371 Date: Jul. 10, 2024

Related U.S. Application Data
(60) Provisional application No. 63/298,532, filed on Jan. 11, 2022.

(51) Int. Cl. A61K 35/28; A61K 51/10; C12N 15/85

(52) U.S. Cl. CPC A61K 35/28; A61K 51/1027; A61K 51/1096; C12N 15/85; A61K 2121/00

(57) ABSTRACT

Provided are methods for treating a CD33-positive hematological malignancy, such as acute myelogenous leukemia or multiple myeloma, in a subject by administering to the subject hematopoietic stem cells (HSCs) or hematopoietic stem and progenitor cells (HSPCs) genetically modified to reduce or eliminate CD33 expression in myeloid cells derived therefrom in conjunction with antibody radioconjugates for one or both of conditioning the subject's bone marrow to receive and engraft the genetically modified stem cells and selectively depleting the subject's endogenous CD33-positive cells, including CD33-positive malignant cells.

### CROSS-REFERENCE TO RELATED APPLICATIONS

This application claims priority to U.S. provisional application Ser. No. 63/298,532 filed Jan. 11, 2022 which is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 9, 2023, is named ATNM-014PCT_SEQ_ST26.xml and is 31,933 bytes in size.

### FIELD OF THE INVENTION

The presently claimed invention relates to the field of oncology.

### BACKGROUND

Overexpression of CD33 is commonly found in many hematological malignancies, including AML, CML, and MDS. In AML, 85-90% of patients express CD33, which has led to the development of targeted therapies. Approximately 96% of MDS patients express CD33 on their myeloblasts (Sanford et al., “CD33 is frequently expressed in cases of myelodysplastic syndrome and chronic myelomonocytic leukemia with elevated blast count,” 2016, Leukemia & Lymphoma, vol. 57 (8): 1965-1968). In another study, MDS patients demonstrated approximately twice as many CD33 molecules per bone marrow cell as the control samples (Jilani, et al., “Differences in CD33 intensity between various myeloid neoplasms,” 2002, Am J Clin Pathol 2002, vol. 118:560-566). The CD33 antigen is expressed on virtually all cases of CML. Moreover, patients older than 60 years have a poor prognosis with only 10% to 15% of 4-year disease-free survival for AML. This high relapse rate for AML patients and the poor prognosis for older patients highlight the urgent need for novel therapeutics preferentially targeting CD33⁺ cells.

U.S. Pat. No. 10,137,155 discloses methods for treating CD33-positive hematological malignancies that involve the administration of hematopoietic stem cells (HSCs) genetically modified to knock out CD33 expression followed by steps to deplete endogenous CD33-expressing hematopoietic cells.

What is needed and provided by the various aspects of the present invention are new and improved methods for treating CD33-positive hematological malignancies using genetically modified hematopoietic stem cells, which methods involve the use of radiolabeled targeting agents such as antibody radioconjugates.

### SUMMARY OF THE INVENTION

One aspect of the invention provides a method for treating a hematological malignancy including CD33-positive malignant cells in a mammalian subject, that includes the steps of.
administering to the subject hematopoietic stem cells (HSCs) or hematopoietic stem and progenitor cells (HSPCs), genetically modified to reduce or eliminate the expression of CD33 antigen by or on myeloid lineage cells that descend from the HSCs or HSPCs, for example, as a result of deletion or disruption of exon 2 of the CD33 gene; and
before and/or after administering the genetically modified HSCs or HSPCs to the subject, administering to the subject an amount of a radiolabeled anti-CD33 antibody effective to kill or inhibit the proliferation of the CD33-positive hematological cells, such as CD33-positive malignant hematological cells, in the subject,
wherein the radiolabeled anti-CD33 antibody is labeled with a radionuclide including ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, ¹⁰³Pd or any combination thereof.
   The administered genetically modified HSCs or HSPCs engraft in the subject and give rise to myeloid lineage cells with reduced or eliminated cell surface CD33 expression; and the administered radiolabeled anti-CD33 antibody kills or inhibits the proliferation of endogenous CD33-positive cells, such as CD33-positive malignant cells, in the subject while at least substantially sparing cells derived from the administered genetically modified HSCs or HSPCs.

Additional features, advantages, and aspects of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

FIG. 1 shows a schematic diagram of expression plasmids for lintuzumab (HuM195). The humanized VL and VH exons of HuM195 are flanked by XbaI sites. The VL exon was inserted into mammalian expression vector pVk, and the VH exon into pVg1 (Co, et al., J. Immunol. 148:1149-1154, 1992).

FIG. 2 shows the complete sequence of the lintuzumab (HuM195) light chain gene cloned in pVk between the XbaI and BamHI sites. The nucleotide number indicates its position in the plasmid pVk-HuM195. The VL and CK exons are translated in single letter code; the dot indicates the translation termination codon. The mature light chain begins at the double-underlined aspartic acid (D). The intron sequence is in italics. The polyA signal is underlined. The full-length nucleotide sequence and the encoded amino acid sequence of the lintuzmab light chain are disclosed as SEQ ID NO:19 and SEQ ID NO:20 respectively.

FIG. 3 shows the complete sequence of the lintuzumab (HuM195) heavy chain gene cloned in pVg1 between the XbaI and BamHI sites. The nucleotide number indicates its position in the plasmid pVg1-HuM195. The VH, CH1, H, CH2 and CH3 exons are translated in single letter code; the dot indicates the translation termination codon. The mature heavy chain begins at the double-underlined glutamine (Q). The intron sequences are in italics. The polyA signal is underlined. The full-length nucleotide sequence and the encoded amino acid sequence of the lintuzumab heavy chain are disclosed as SEQ ID NO:21 and SEQ ID NO:22 respectively.

FIG. 4 shows a process for making ²²⁵Ac-labeled lintuzumab (²²⁵Ac-HuM195) that involves reacting p-SCN-Bn-DOTA with lintuzumab to form a conjugate followed by chelation of ²²⁵Ac to the DOTA moiety of the conjugate.

FIG. 5 shows a flowchart for the production of ²²⁵Ac-labeled lintuzumab (²²⁵Ac-HuM195).

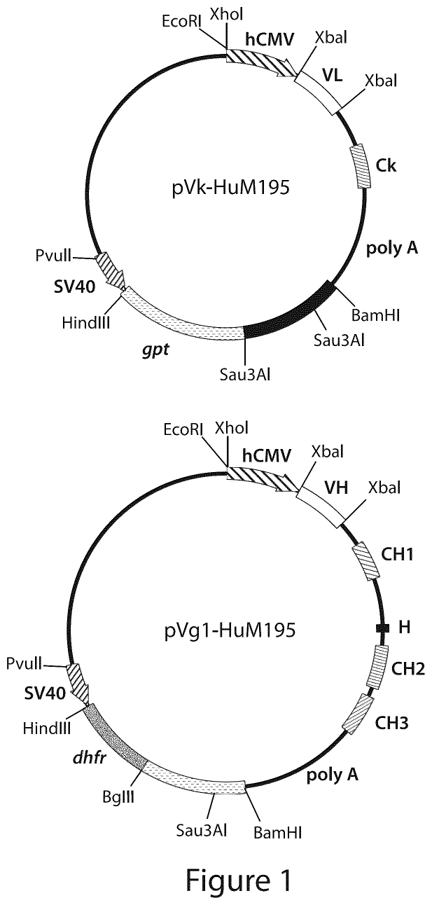

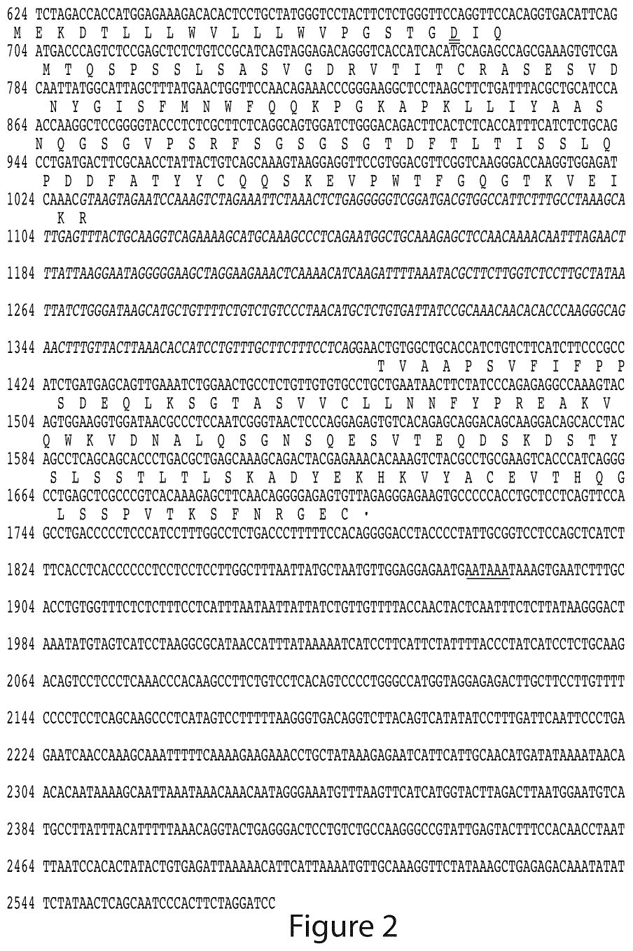

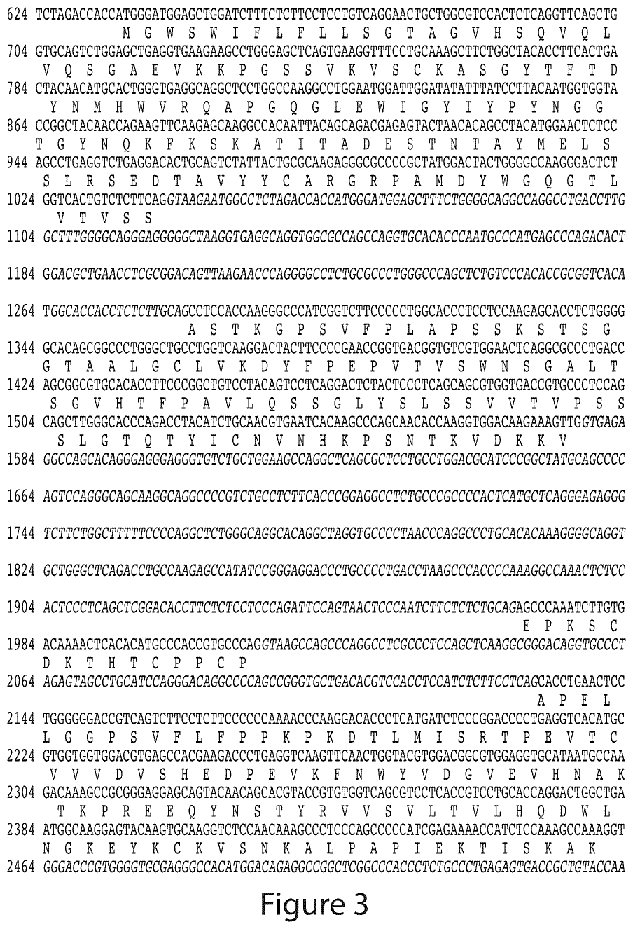

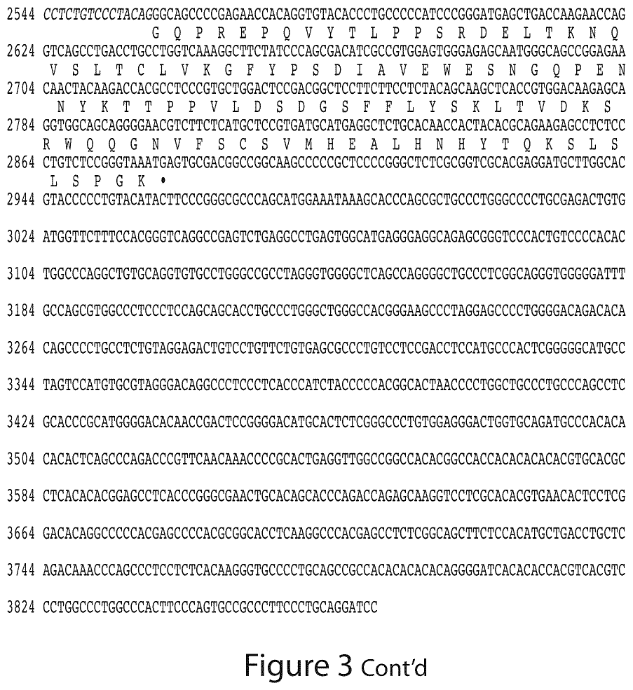

### DETAILED DESCRIPTION

In one aspect, the presently disclosed invention provides compositions and methods for treating a CD33-positive hematological malignancy in a mammalian subject, such as a human patient, by administering to the subject hematopoietic stem cells (HSCs) or hematopoietic stem and progenitor cells (HSPCs) genetically modified to reduce or eliminate CD33 expression in myeloid cells derived therefrom in conjunction with administering antibody radioconjugates for one or both of conditioning the subject's bone marrow to receive and engraft the genetically modified stem cells using, for example, a radiolabeled anti-CD45 antibody such as but not limited to ¹³¹I-labeled or ²²⁵Ac-labeled apamistamab (BC8), and selectively depleting the subject's endogenous CD33-positive cells, including CD33-positive malignant cells, using, for example, a radiolabeled anti-CD33 antibody, such as but not limited to ²²⁵Ac-labeled linzutumab (HuM195).

Prior to administering the genetically modified HSCs or HSPCs, the subject may, for example, be treated with a myeloablative conditioning regimen with the aim of completely depleting both their endogenous HSCs and as well as any endogenous hematological cancer cells, before and in preparation of administration of the genetically modified HSCs or HSPCs to the subject. Endogenous CD33-expressing hematological cells that remain or recur following the HSC or HSPC transplantation, whether malignant or non-malignant, may be selectively killed by administering a radiolabeled CD33 targeting agent, such as ²²⁵Ac-labeled lintuzumab, to the subject.

The CD33-positive hematological malignancy treated by the methods of the invention may, for example, be multiple myeloma (MM), acute myeloid leukemia (AML), chronic myelogenous leukemia (CML), myelodysplastic syndrome (MDS), or a myeloproliferative neoplasm such as chronic myelomonocytic leukemia (CMML). The hematological malignancy may, for example, be a relapsed and/or refractory (R/R) form or occurrence of: multiple myeloma, acute myeloid leukemia, chronic myelogenous leukemia, myelodysplastic syndrome, or a myeloproliferative neoplasm such as CMML. The hematological malignancy may, for example, be refractory and/or resistant to treatment with a Bel-2 inhibitor, such as venetoclax.

As used herein, the term “subject” includes, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission. The human subject may, for example, be relapsed from a prior bone marrow transplant (BMT) or hematopoietic stem cell transplant (HSCT) intended to treat the cancer. The human subject may, for example, be relapsed or refractory to at least one, such as one or two, prior regimens of the same or different therapeutic regimens for the treatment of the cancer.

In general, the radiolabeled targeting agents, such as monoclonal antibodies or antigen-binding fragments thereof, used in the various aspects of the invention may be labeled with one or more of the following radionuclides: ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, and ¹⁰³Pd. Radioisotopes of Iodine may, for example, be chemically conjugated to the targeting agent. For other radionuclides, such as ²²⁵Ac and ¹⁷⁷Lu, it is convenient to chemically conjugate a chelator (chelator moiety), such as DOTA or a derivative thereof, to the targeting agent and radiolabel the targeting agent by chelation of the radionuclide to the conjugated chelator.

The chelator group in the various aspects of the invention may, for example, include: 1,4,7,10-tetraazacyclododecane-1,4,7-triacetic acid (DO3A) or a derivative thereof; 1,4,7-triazacyclononane-1,4-diacetic acid (NODA) or a derivative thereof; 1,4,7-triazacyclononane-1,4,7-triacetic acid (NOTA) or a derivative thereof; 1,4,7,10-tetraazacyclododecane-1,4,7,10-tetraacetic acid (DOTA) or a derivative thereof; 1,4,7-triazacyclononane, 1-glutaric acid-4,7-diacetic acid (NODAGA) or a derivative thereof; 1,4,7,10-tetraazacyclodecane, 1-glutaric acid-4,7,10-triacetic acid (DOTAGA) or a derivative thereof; 1,4,8,11-tetraazacyclotetradecane-1,4,8,11-tetraacetic acid (TETA) or a derivative thereof; 1,4,8,11-tetraazabicyclo[6.6.2]hexadecane-4,11-diacetic acid (CB-TE2A) or a derivative thereof; diethylene triamine pentaacetic acid (DTPA), its diester, or a derivative thereof, 2-cyclohexyl diethylene triamine pentaacetic acid (CHX-A″-DTPA) or a derivative thereof; deforoxamine (DFO) or a derivative thereof; 1,2-[[6-carboxypyridin-2-yl]methylamino]ethane (H₂dedpa) or a derivative thereof; DADA or a derivative thereof; 1,4,7,10-Tetraazacyclododecane-1,4,7,10-tetra(methylene phosphonic acid) (DOTP) or a derivative thereof; 4-amino-6-[[16-[(6-carboxypyridin-2-yl)methyl]-1,4,10,13-tetraoxa-7,16-diazacyclooctadec-7-yl]methyl]pyridine-2-carboxylic acid (MACROPA-NH₂) or a derivative thereof; MACROPA or a derivative thereof; 1,4,7,10-tetrakis(carbamoylmethyl)-1,4,7,10-tetraazacyclododecane (TCMC) or a derivative thereof; {4-[2-(bis-carboxymethylamino)-ethyl]-7-carboxymethyl-[1,4,7]triazonan-1-yl}-acetic acid (NETA) or a derivative thereof; Diamsar or a derivative thereof; 1,4,7-triazacyclononane-1,4,7-tris[methyl (2-carboxyethyl)phosphinic acid (TRAP, PRP9, TRAP-Pr) or a derivative thereof; N,N′-bis(6-carboxy-2-pyridylmethyl)ethylenediamine-N,N′-diacetic acid (H4octapa) or a derivative thereof; N,N′-[1-benzyl-1,2,3-triazole-4-yl]methyl-N,N′-[6-(carboxy)pyridin-2-yl]-1,2-diaminoethane (H2azapa) or a derivative thereof; N,N″-[[6-(carboxy)pyridin-2-yl]methyl]diethylenetriamine-N,N′,N″-triacetic acid (H5decapa) or a derivative thereof; N,N′-bis(2-hydroxy-5-sulfobenzyl)ethylenediamine-N,N′-diacetic acid (SHBED) or a derivative thereof; N,N′-bis(2-hydroxybenzyl)ethylenediamine-N,N′-diacetic acid (HBED) or a derivative thereof; 3,6,9,15-tetraazabicyclo [9.3.1]pentadeca-1 (15),11,13-triene-3,6,9-triacetic acid (PCTA) or a derivative thereof; desferrioxamine B (DFO) or a derivative thereof; N,N′-(methylenephosphonate)-N,N′-[6-(methoxycarbonyl)pyridin-2-yl]methyl-1,2-diaminoethane (H6phospa) or a derivative thereof; 1,4,7,10,13,16-hexaazacyclohexadecane-N,N′,N″,N′″,N″″,N′″″-hexaacetic acid (HEHA) or a derivative thereof; 1,4,7,10,13-pentaazacyclopentadecane-N,N′,N″,N′″,N″″-pentaacetic acid (PEPA) or a derivative thereof; or 3,4,3-LI (1,2-HOPO) or a derivative thereof.

The chelator group in the various aspects of the invention may, for example, include a chelator group selected from:

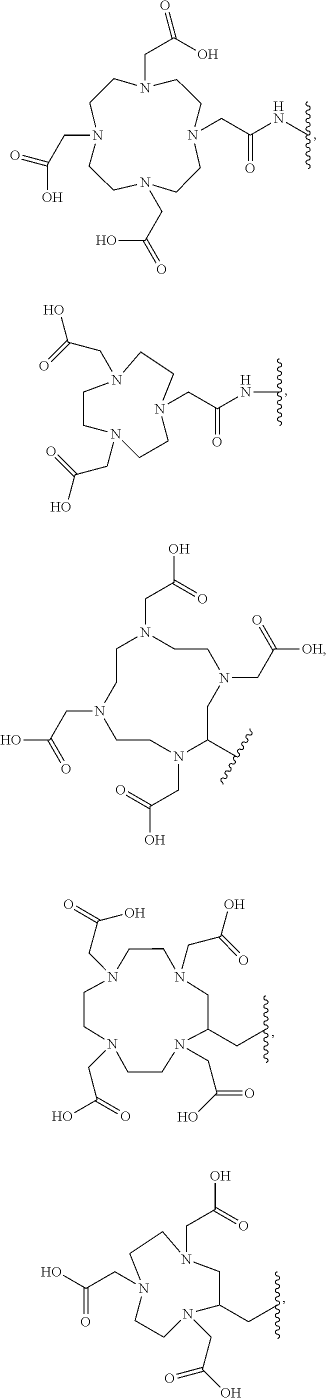 [SMILES: *NC(=O)CN1CCN(CC(=O)O)CCN(CC(=O)O)CCN(CC(=O)O)CC1 ; *NC(=O)CN1CCN(CC(=O)O)CCN(CC(=O)O)CC1 ; *C1CN(CC(=O)O)CCN(CC(=O)O)CCN(CC(=O)O)CCN1CC(=O)O ; *CC1CN(CC(=O)O)CCN(CC(=O)O)CCN(CC(=O)O)CCN1CC(=O)O ; *CC1CN(CC(=O)O)CCN(CC(=O)O)CCN1CC(=O)O]

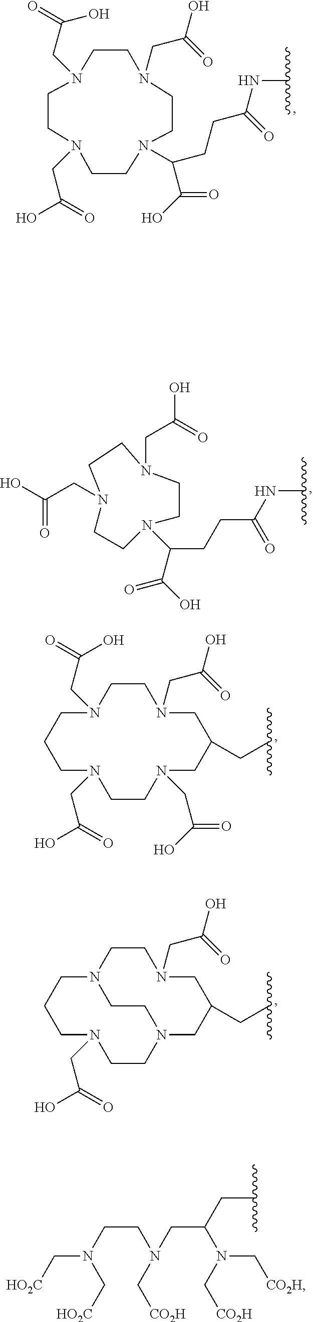

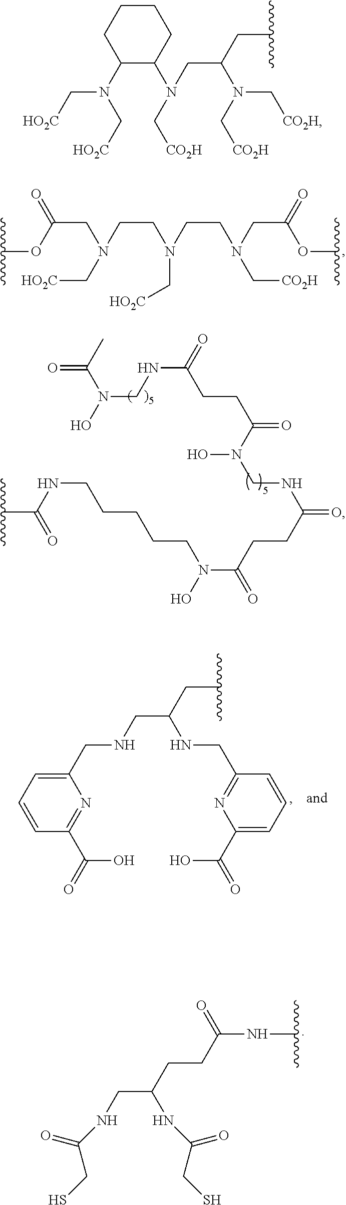

The CD33 targeting and other antigen targeting agents employed in the various aspects of the invention may, for example, be antibodies or antigen-binding fragments thereof such as any of the types described herein.

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, scFv, diabodies, minibodies, and nanobodies (sdAb); (d) naturally occurring and non-naturally occurring, such as wholly synthetic antibodies, IgG-Fc-silent, and chimeric; and (e) bi-specific forms thereof. Immunoglobulin molecules may, for example, derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies may be human, humanized or nonhuman. When a specific aspect of the presently disclosed invention refers to or recites an “antibody,” it is envisioned as referring to any of the full-length antibodies or antigen-binding fragments thereof disclosed herein, unless explicitly denoted otherwise.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

Compositions including a radiolabeled antibody or radiolabeled antigen-binding antibody fragment may include one or more pharmaceutically acceptable carriers or pharmaceutically acceptable excipients. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's). An exemplary formulation may be as substantially described in International Pub. No. WO 2017/155937, incorporated by reference herein. For example, according to certain aspects, the formulation may include 0.5% to 5.0% (w/v) of an excipient selected from the group consisting of ascorbic acid, polyvinylpyrrolidone (PVP), human serum albumin (HSA), a water-soluble salt of HSA, and mixtures thereof. Certain formulations may include 0.5-5% ascorbic acid; 0.5-4% polyvinylpyrrolidone (PVP); and the monoclonal antibody in 50 mM PBS buffer, pH 7.

Although throughout the present disclosure various aspects or elements thereof are described in terms of “including” or “comprising,” it should be understood that corresponding aspects or elements thereof described in terms of “consisting essentially of” or “consisting of” are similarly disclosed and provided by this disclosure. For example, while certain aspects of the invention have been described in terms of a method “including” or “comprising” administering a radiolabeled antibody, corresponding methods instead reciting “consisting essentially of” or “consisting of” administering the radiolabeled antibody are also within the scope of said aspects and disclosed by this disclosure.

Hematopoietic stem cells (HSCs) or hematopoietic stem and progenitor cells (HSPCs) genetically modified to knockout the CD33 gene and/or its expression and methods for producing such cells, which may be used in the implementation of the various embodiments of the present invention, are known in the art and described, for example, in each of U.S. Pat. Nos. 10,925,902, 10,137,155, U.S. Pub. No. 20210252073, U.S. Pub. No. 202102600130 and International Pub No. WO2020172638A1. Such cells for genetic modification are CD34⁺ CD33⁻ and may, for example, be obtained from cord blood or from bone marrow (e.g., from G-CSF-mobilized stem cells) as known in the art. See AbuSamra et al., Not just a marker: CD34 on human hematopoietic stem/progenitor cells dominates vascular selectin binding along with CD44. Blood Adv. 2017; 1 (27): 2799-2816. Published 2017 Dec. 26. Doi: 10.1182/bloodadvances.2017004317. Once isolated, the stem cells can be genetically modified and expanded according to methods known in the art. The cells that are modified and used to treat a subject may be autologous, syngeneic, or allogeneic in origin. A single and/or total dose of genetically modified HSCs or HSPCs administered to the subject may, for example, be at least 1×10⁶ cells/kg (subject weight), at least 2×10⁶ cells/kg, at least 3×10⁶ cells/kg, at least 4×10⁶ cells/kg, at least 5×10⁶ cells/kg, at least 10×10⁶ cells/kg, 1-50×10⁶ cells/kg or any subrange of cells/kg between interval values in said range or any integer value of cells/kg in said range, such as 2-30×10⁶ cells/kg, 2-20×10⁶ cells/kg, or 1-30×10⁶ cells/kg.

An antibody drug conjugate (ADC) approach to eliminating residual CD33 hematological cells following transplantation of CD33 knock-out HSCs or HSPCs, using the anti-CD33 ADC gemtuzumab ozogamicin (Mylotarg®; Pfizer, Inc.), is being developed by Vor Biopharma Inc. (Cambridge, MA USA) for the treatment of AML in human patients. However, resistance to the drug payload of gemtuzumab ozogamicin has been described and the activity of the agent depends on its internalization by CD33-expressing cells which can vary in their ability to internalize the agent. In contrast, the radiolabeled CD33-targeting agents employed in aspects of the present invention are not subject to payload-specific resistance mechanisms and do not require internalization by CD33-expressing cells or high levels of CD33 cell surface expression for depletion of said cells. Most particularly, radiolabeled CD33 targeting agents labeled with alpha particle emitting radionuclide, such as ²²⁵Ac, ²²⁷Th, ²¹³Bi or ²¹¹At, emit high energy alpha particles that cause double stranded DNA breaks that cannot be overcome by resistance mechanisms. Accordingly, in one aspect of the present invention, following transplantation of CD33 knock-out (negative) HSCs or HSPCs to a patient in need of treatment for a CD33-expressing hematological malignancy, a radiolabeled CD33 targeting agent, such as ²²⁵Ac-labeled lintuzumab, is administered to the patient to deplete (or ensure depletion of) any residual CD33-expressing hematological cells in the patient, rather than using a CD33 targeting ADC, such as gemtuzumab ozogamicin. In another aspect of the invention, following transplantation of CD33 knock-out HSCs or HSPCs to a patient in need of treatment for a CD33-expressing hematological malignancy, a CD33 targeting ADC such as gemtuzumab ozogamicin is administered to the patient to deplete residual CD33-expressing hematological cells in the patient and a radiolabeled CD33 targeting agent, such as ²²⁵Ac-labeled lintuzumab, is administered to the patient to deplete (or ensure depletion of) residual CD33-expressing hematological cells in the patient. In one variation, the ADC such as gemtuzumab ozogamicin is administered to the patient first, and at a later time, for example, at least one, two, three or four weeks later, the radiolabeled CD33 targeting agent is administered to the patient. In a further variation, a step of detecting any residual CD33-expressing hematological cells is performed following administration/treatment with the CD33 targeting ADC and if such CD33-expressing cells are detected, the radiolabeled CD33 targeting agent is administered to the patient to deplete such remaining/residual CD33 expressing cells. Thus, the radiolabeled CD33 targeting agent may, for example, be used to deplete residual CD33-expressing hematological cells that are refractory to complete/full depletion by the CD33 targeting ADC.

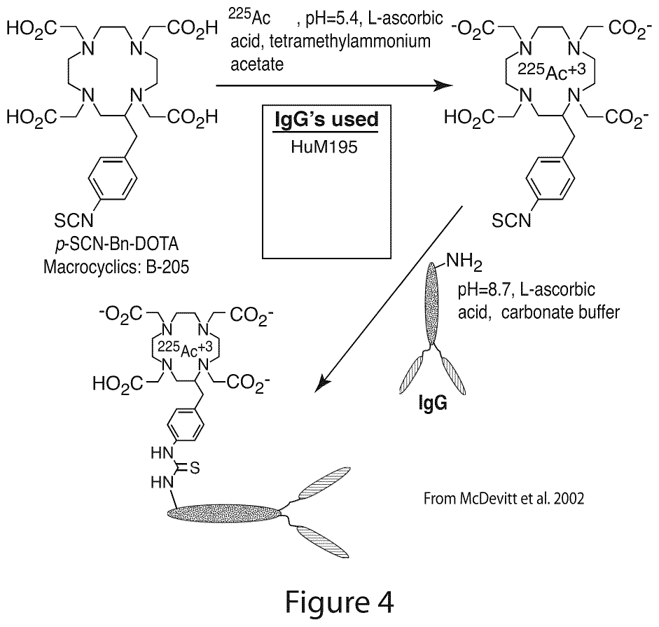

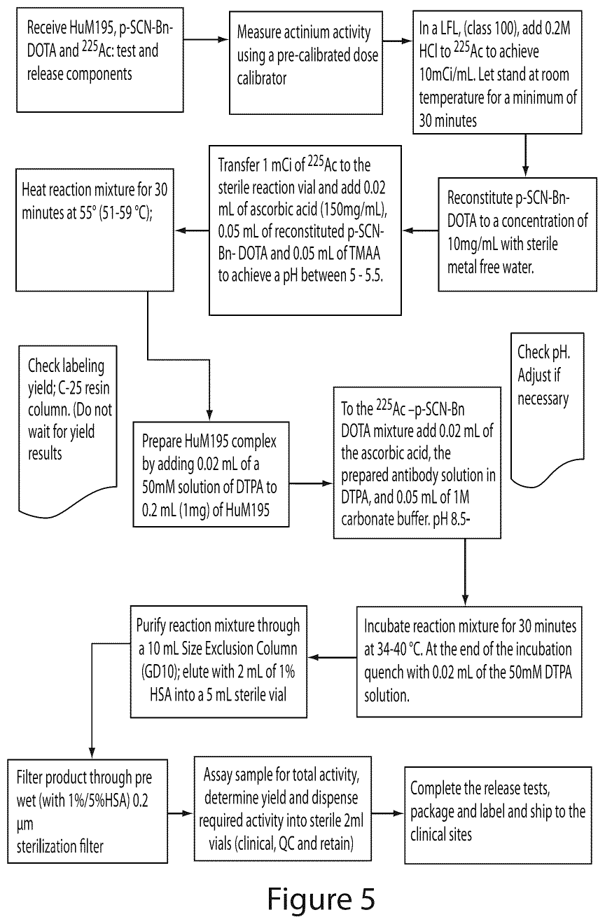

FIGS. 4 and 5 show an exemplary process and workflow for making ²²⁵Ac-labeled lintuzumab which may be used to prepare ²²⁵Ac-labeled lintuzumab for use in the various aspects of the invention. Methods for preparing ²²⁵Ac-labeled radioimmunoconjugates, including ²²⁵Ac-labeled lintuzumab (HuM195) and pharmaceutical compositions thereof, are further disclosed, for example, in Applicant's U.S. Pat. No. 9,603,954, which methods may be used to produce ²²⁵Ac-labeled anti-CD33 antibodies and/or ²²⁵Ac-labeled anti-CD45 antibodies, and respective pharmaceutical compositions thereof, for use in the various aspects of the present invention. The full-length nucleotide sequence and the encoded amino acid sequence of the lintuzumab light chain are disclosed as SEQ ID NO: 19 and SEQ ID NO:20 respectively. The full-length nucleotide sequence and the encoded amino acid sequence of the lintuzumab heavy chain are disclosed as SEQ ID NO:21 and SEQ ID NO:22 respectively.

The therapeutic methods of the invention may include a conditioning regimen prior to administration of the genetically modified HSCs or HSPCs to the subject to facilitate their engraftment in the recipient. Such a conditioning regimen may, for example, be myeloablative, non-myeloablative, or reduced intensity conditioning (RIC) or otherwise intermediate intensity between myeloablative and non-myeloablative condition. An exemplary conditioning regimen that may be used is a conditioning regimen for autologous peripheral blood stem cell transplant (PBSCT) consisting of etoposide (VP-16) at 1.8 g/m² i.v. constant infusion (c.i.v.) over 26 h for 3 days, followed by total body irradiation (TBI) at 300 cGy per day for the next 3 days.

A conditioning regimen/treatment use in connection with the methods of treatment disclosed herein may, for example, include or consist of administration of a radiolabeled anti-CD45 targeting agent, such as a radiolabeled anti-CD45 monoclonal antibody. The anti-CD45 antibody may, for example, be apamistamab (also known as BC8), an antibody including the heavy chain variable region and/or the light chain variable region of apamistamab, an antibody including the heavy chain CDRs and/or the light chain CDRs of apamistamab, an antibody including a light chain including the amino acid sequence set forth SEQ ID NO: 16 and a heavy chain including the amino acid sequence set forth in SEQ ID NO:17 or SEQ ID NO:18, a humanized form of apamistamab, a chimeric antibody form of apamistamab (for example, including a human IgG Fc region), or an antigen-binding fragment of any of the aforementioned antibodies such as a Fab fragment or a Fab₂ fragment thereof.

An exemplary myeloablative conditioning regimen that may be employed in various aspects of the present invention includes administration of ¹³¹I-apamistamab (Iomab-B™, Actinium Pharmaceuticals, Inc., New York, NY USA) as a single dose in an amount delivering a maximum dose to the liver of 24 Gy (e.g., a mean amount of approximately 600 mCi), fludarabine (30 mg/m2 patient surface area×3 days) and total body irradiation (TBI) of 2 Gy, with administration of the genetically modified HSCs or HSCPCs performed 12-14 days after administration of ¹³¹I-apamistamab. In a related example, the myeloablative conditioning regimen includes or consists of administration of ¹³¹I-apamistamab as a single dose in an amount delivering a maximum radiation dose to the liver of 24 Gy (such as an amount of approximately 600 mCi) on day −12 (with respect to HSCT performed on day 0), fludarabine (30 mg/m² patient surface area) on each of days −4, −3 and −2, and 2 Gy TBI on day 0 prior to administration of the genetically modified HSCs or HSPCs on the same day.

Methods for conditioning a mammalian subject such as a human to facilitate engraftment and/or activity of cells administered as an adoptive cell therapy and/or as a gene-edited cell-based therapy using radiolabeled anti-CD45 monoclonal antibodies, which may be employed in the various embodiments of the present invention are described, for example, in Applicant's U.S. Pub. Nos. 20200308280A1, 20200255520A1, and 20200283539A1. Stabilized pharmaceutical compositions including ¹³¹I-labeled anti-CD45 monoclonal antibody that may be employed in the various aspects of the present invention are disclosed, for example, in Applicant's U.S. Pat. No. 10,420,851. ²²⁵Ac and ¹⁷⁷Lu-labeled anti-CD45 antibodies that may be employed in various aspects of the present invention are disclosed, for example, in Applicant's International Pub. No. WO2021055638 (International App. No. PCT/US2020/051324).

SEQ ID NO: 1 provides the amino acid sequence of the variable domain of the light chain of anti-CD45 murine immunoglobulin apamistamab.

SEQ ID NO:2 provides the amino acid sequence of the variable domain of the heavy chain of anti-CD45 murine immunoglobulin apamistamab.

SEQ ID NO:3 provides the amino acid sequence of CDR1 of the light chain of anti-CD45 murine immunoglobulin apamistamab.

SEQ ID NO:4 provides the amino acid sequence of CDR2 of the light chain of anti-CD45 murine immunoglobulin apamistamab.

SEQ ID NO:5 provides the amino acid sequence of CDR3 of the light chain of anti-CD45 murine immunoglobulin apamistamab.

SEQ ID NO:6 provides the amino acid sequence of CDR1 of the heavy chain of anti-CD45 murine immunoglobulin apamistamab.

SEQ ID NO: 7 provides the amino acid sequence of CDR2 of the heavy chain of anti-CD45 murine immunoglobulin apamistamab.

SEQ ID NO:8 provides the amino acid sequence of CDR3 of the heavy chain of anti-CD45 murine immunoglobulin apamistamab.

SEQ ID NO:9 provides the nucleotide sequence of the light chain of anti-CD45 murine immunoglobulin apamistamab.

SEQ ID NO: 10 provides the nucleotide sequence of the heavy chain of anti-CD45 murine immunoglobulin apamistamab.

SEQ ID NO: 11 provides the amino acid sequence of N-terminus of the light chain of anti-CD45 murine immunoglobulin apamistamab.

SEQ ID NO: 12 provides the amino acid sequence of N-terminus of the heavy chain of anti-CD45 murine immunoglobulin apamistamab.

SEQ ID NO: 13 provides the predicted amino acid sequence of the light chain of anti-CD45 murine immunoglobulin apamistamab.

SEQ ID NO:14 provides the predicted amino acid sequence of the heavy chain of anti-CD45 murine immunoglobulin apamistamab.

SEQ ID NO: 15 provides the actual amino acid sequence of the heavy chain of anti-CD45 murine immunoglobulin apamistamab as determined by amino-acid sequencing.

SEQ ID NO: 16 provides the predicted amino acid sequence of the light chain of anti-CD45 murine immunoglobulin apamistamab, excluding N-terminal leader sequence.

SEQ ID NO: 17 provides the predicted amino acid sequence of the heavy chain of anti-CD45 murine immunoglobulin apamistamab, excluding N-terminal leader sequence.

SEQ ID NO:18 provides the actual amino acid sequence of the heavy chain of anti-CD45 murine immunoglobulin apamistamab as determined by amino-acid sequencing, excluding N-terminal leader sequence.

Without limitation, the invention further provides the following enumerated embodiments/aspects:

Embodiment 1. A method for treating a hematological malignancy including CD33-positive malignant cells in a mammalian subject, including the steps of:
administering to the subject hematopoietic stem cells (HSCs) or hematopoietic stem and progenitor cells (HSPCs), genetically modified to reduce or eliminate the expression of CD33 antigen by or on myeloid lineage cells that descend from the HSCs or HSPCs, for example, as a result of deletion or disruption of exon 2 of the CD33 gene; and
before and/or after administering the genetically modified HSCs or HSPCs to the subject, administering to the subject an amount of a radiolabeled anti-CD33 antibody effective to kill or inhibit the proliferation of the CD33-positive malignant cells in the subject, wherein the radiolabeled anti-CD33 antibody is labeled with a radionuclide including ¹³¹I,
¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, ¹⁰³Pd or any combination thereof.

Embodiment 2. The method of embodiment 1, wherein
the administered genetically modified HSCs or HSPCs engraft in the subject and give rise to myeloid lineage cells with reduced or eliminated cell surface CD33 expression; and
the administered radiolabeled anti-CD33 antibody kills or inhibits the proliferation of endogenous CD33-positive cells, such as CD33-positive malignant cells, in the subject.

Embodiment 3. The method of embodiment 1 or 2, wherein the radiolabeled anti-CD33 antibody includes radiolabeled lintuzumab (HuM195), radiolabeled gemtuzumab, radiolabeled vadastuximab, a radiolabeled antibody comprising the heavy chain complementarity determining regions (CDRs) and/or the light chain CDRs of any of the preceding antibodies, a radiolabeled antibody comprising the heavy chain variable region and/or the light chain variable region of any of the preceding antibodies or a radiolabeled antigen-binding fragment of any of the preceding antibodies such as a Fab fragment or a Fab₂ fragment thereof.

Embodiment 4. The method of any one of preceding embodiments, wherein the radiolabeled anti-CD33 antibody is radiolabeled with ²²⁵Ac or ¹⁷⁷Lu.

Embodiment 5. The method of embodiment 4, wherein the radiolabeled anti-CD33 antibody is chemically conjugated to a chelator including 1,4,5,10-tetraazacyclododecance-1,4,7,10-tetracetic acid (DOTA) or a derivative thereof, and is labeled with the radionuclide ²²⁵Ac or ¹⁷⁷Lu by chelation of the radionuclide by the conjugated chelator.

Embodiment 6. The method of any one of the preceding embodiments, wherein the radiolabeled anti-CD33 antibody is ²²⁵Ac-labeled and the effective amount of the ²²⁵Ac-labeled antibody includes a dose of 0.1 to 10 μCi/kg subject body weight.

Embodiment 7. The method of any one of the preceding embodiments, wherein the radiolabeled anti-CD33 antibody is ²²⁵Ac-labeled and the effective amount of the ²²⁵Ac-labeled antibody includes a dose of 0.5 to 4 μCi/kg subject body weight.

Embodiment 8. The method of any one of the preceding embodiments, wherein the step of administering the radiolabeled anti-CD33 antibody is performed after the step of administering the HSCs or HSPCs genetically modified to reduce or eliminate the expression of CD33 antigen by or on myeloid lineage cells that descend from said HSCs or HSPCs.

Embodiment 9. The method of any one of the preceding embodiments, wherein the step of administering the radiolabeled anti-CD33 antibody is performed before the step of administering the genetically modified HSCs or HSPCs.

Embodiment 10. The method of any one of the preceding embodiments, further including the step of:
before administering the HSCs or HSPCs genetically modified to reduce or eliminate the expression of CD33 antigen by or on myeloid lineage cells that descend from the hematopoietic stem cells,
administering an amount of a radiolabeled anti-CD45 antibody effective to facilitate engraftment of genetically modified HSCs or HSPCs pursuant to their administration.
   wherein the radiolabeled anti-CD45 antibody is labeled with a radionuclide including ¹³¹I, ¹²⁵I, ¹²³I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, ¹⁰³Pd or any combination thereof.

In one variation of this embodiment, administration of the radiolabeled anti-CD45 antibody is part of a myeloablative conditioning regimen administered to the subject prior to and in preparation of administering the genetically modified HSCs or HSPCs to the subject. Such a myeloablative regimen may, for example, include administration of 131-Iodine labeled anti-CD45 antibody, such as ¹³¹I-apamistamab, followed by administration of fludarabine, followed by total body irradiation.

Embodiment 11. The method of embodiment 10, wherein the radiolabeled anti-CD45 antibody is labeled with ¹³¹I, ¹⁷⁷Lu, or ²²⁵Ac.

Embodiment 12. The method of embodiment 11, wherein radiolabeled anti-CD45 antibody is labeled with ¹³¹I, and the amount administered is selected from the group consisting of from 10 mCi to 200 mCi, from 200 mCi to 400 mCi, and from 400 mCi to 1,200 mCi.

Embodiment 13. The method of embodiment 11, wherein the radiolabeled anti-CD45 antibody is labeled with ²²⁵Ac, the amount administered is selected from the group consisting of from 0.1 μCi/kg to 5.0 μCi/kg subject weight, from 0.1 μCi/kg to 1.0 μCi/kg subject weight, from 1.0 μCi/kg to 3.0 μCi/kg subject weight, and from 3.0 μCi/kg to 5.0 μCi/kg subject weight.

Embodiment 14. The method of embodiment 11, wherein the radiolabeled anti-CD45 antibody is labeled with ¹³¹I, and administered in an amount selected from the group consisting of: from 10 mCi to 200 mCi administered 6, 7, or 8 days before administering the genetically modified HSCs or HSPCs to the subject, from 200 mCi to 400 mCi administered 8, 9, 10, 11, or 12 days before administering the genetically modified HSCs or HSPCs to the subject; and from 400 mCi to 1,200 mCi of administered 10, 11, 12, 13, or 14 days before administering the genetically modified HSCs or HSPCs to the subject.

Embodiment 15. The method of embodiment 11, wherein the radiolabeled anti-CD45 antibody is labeled with ²²⁵Ac, and administered in an amount from 0.1 μCi/kg to 5.0 μCi/kg subject weight administered 6, 7, 8, 9, 10, 11, or 12 days before administering the genetically modified HSCs or HSPCs to the subject.

Embodiment 16. The method of any one of embodiments 10-15, wherein the radiolabeled anti-CD45 antibody includes heavy chain CDRs (HC-CDRs) having the following amino acid sequences and/or the light chain CDRs (LC-CDRs) having the following amino acid sequences
LC-CDR-1: RASKSVSTSGYSYLH (SEQ ID NO:3)
LC-CDR-2: LASNLES (SEQ ID NO:4)
LC-CDR-3: QHSRELPFT (SEQ ID NO:5)
HC-CDR-1: GFDFSRYWMS (SEQ ID NO:6)
HC-CDR-2: EINPTSSTINFTPSLKD (SEQ ID NO:7)
HC-CDR-3: GNYYRYGDAMDY (SEQ ID NO:8).

Embodiment 17. The method of embodiment 16, wherein the radiolabeled antibody includes radiolabeled apamistamab or an antigen-binding fragment thereof.

Embodiment 18. The method of any one of the embodiments 10-17, wherein the step of administering to the subject an amount of a radiolabeled anti-CD45 antibody including administering to the subject a composition including a radiolabeled fraction of the anti-CD33 antibody and a non-radiolabeled fraction of the anti-CD33 antibody in a ratio of 0.1:1 to 1:1 radiolabeled to non-radiolabeled.

Embodiment 19. The method of embodiment 18, wherein the amount of the anti-CD45 antibody in the composition administered is less than 16 mg/kg of subject body weight.

Embodiment 20. The method of any one of the preceding embodiments, wherein the step of administering to the subject an amount of a radiolabeled anti-CD33 antibody includes administering to the subject a composition including a radiolabeled fraction of the anti-CD33 antibody and a non-radiolabeled fraction of the anti-CD33 antibody in a ratio of 0.1:1 to 1:1 radiolabeled to non-radiolabeled.

Embodiment 21. The method of embodiment 20, wherein the amount of the anti-CD33 antibody in the composition administered is less than 16 mg/kg of subject body weight.

Embodiment 22. The method of any one of the preceding embodiments, wherein the hematological malignancy is multiple myeloma, acute myeloid leukemia (AML), chronic myelogenous leukemia (CML), myelodysplastic syndrome (MDS), chronic myelomonocytic leukemia (CMML), or a myeloproliferative neoplasm.

Embodiment 23. The method of embodiment of any one of the preceding embodiments, wherein the hematological malignancy is a relapsed and/or refractory malignancy.

Embodiment 24. The method of any one of the preceding embodiments, wherein the mammalian subject is a human patient.

Embodiment 25. The method of any one of the preceding embodiments, wherein no conditioning agent other than a radiolabeled anti-CD45 antibody is administered to the subject prior to administration of the genetically modified HSCs or HSPCs to the subject.

Embodiment 26. The method of any one of the preceding embodiments, wherein no external radiation is administered to the subject prior to and in preparation of the administration of the genetically modified HSCs or HSPCs to the subject.

Embodiment 27. The method of any one of embodiments 1-24, wherein prior to administration of the genetically modified HSCs or HSPCs to the subject, the subject is treated with a myeloablative conditioning regimen.

Embodiment 28. The method of embodiment 27, wherein the myeloablative conditioning regimen includes administration of a radiolabeled anti-CD45 antibody or radiolabeled CD45-binding antibody fragment to the subject.

Embodiment 29. The method of embodiment 27, wherein the myeloablative conditioning regimen includes administration of a radiolabeled human CD45 targeting agent, wherein the targeting agent is a human CD45 binding antibody or antibody fragment including the immunoglobulin heavy chain CDRs and immunoglobulin light chain CDRs of apamistamab, for example, apamistamab itself or a chimeric or humanized form of apamistamab.

Embodiment 30. The method of any one of the preceding embodiments, wherein CD33-targeted cell therapy directed against the subject's endogenous CD33-positive cells is not performed.

Embodiment 31. The method of any one of the preceding embodiments, wherein an anti-CD33 antibody drug conjugate is not administered to the subject after administration of the genetically modified HSCs or HSPCs to the subject.

Embodiment 32. The method of any one of embodiments 1-30, wherein an anti-CD33 antibody drug conjugate is not administered to the subject before, during or after administration of the genetically modified HSCs or HSPCs to the subject.

Embodiment 33. The method of any one of embodiments 1-30, further including the step of administering an anti-CD33 antibody drug conjugate, such as gemtuzumab ozogamicin, to the subject after administration of the genetically modified HSCs or HSPCs to the subject.

Embodiment 34. The method of embodiment 33, wherein after the step of administering the genetically modified HSCs or HSPCs to the subject, one or more steps of administering an anti-CD33 antibody drug conjugate, such as gemtuzumab ozogamicin, to the subject are performed, before administering to the subject an amount of a radiolabeled anti-CD33 antibody effective to kill or inhibit the proliferation of CD33-positive endogenous cells, such as CD33-positive malignant cells, in the subject. In one variation of this embodiment, the method does not include a step of administering a CD33 targeting agent, such as an anti-CD33 antibody, anti-CD33 ADC, anti-CD33 antibody radioconjugate, or CD33-expressing-cell-depleting cell therapy, such as a CD33 targeted CAR-T therapy, before administering the genetically modified HSCs or HSPCs to the subject.

Embodiment 35. The method of any one of the preceding embodiments, wherein CD33-expressing-cell-depleting cell therapy, such as a CD33 targeted CAR-T therapy, is not administered to the subject after administration of the genetically modified HSCs or HSPCs to the subject.

Embodiment 36. The method of any one of embodiments 1-30, wherein a CD33-expressing-cell-depleting cell therapy, such as a CD33 targeted CAR-T therapy, is not administered to the subject before, during or after administration of the genetically modified HSCs or HSPCs to the subject.

Embodiment 37. The method of any one of embodiments 1-30, further including the step of administering a CD33-expressing-cell-depleting cell therapy, such as a CD33 targeted CAR-T therapy to the subject after administration of the genetically modified HSCs or HSPCs to the subject.

Embodiment 38. The method of embodiment 37, wherein after the step of administering the genetically modified HSCs or HSPCs to the subject, one or more steps of administering a CD33-expressing-cell-depleting cell therapy, such as a CD33 targeted CAR-T therapy, to the subject are performed, before administering to the subject an amount of a radiolabeled anti-CD33 antibody effective to kill or inhibit the proliferation of CD33-positive endogenous cells, such as CD33-positive malignant cells, in the subject. In one variation of this embodiment, the method does not include a step of administering a CD33 targeting agent, such as an anti-CD33 antibody, anti-CD33 ADC, anti-CD33 antibody radioconjugate or CD33-expressing-cell-depleting cell therapy, such as a CD33 targeted CAR-T therapy, before administering the genetically modified HSCs or HSPCs to the subject.

Embodiment 39. The method of any one of the preceding embodiments, wherein the step of administering to the subject an amount of a radiolabeled anti-CD33 antibody effective to kill or inhibit the proliferation of endogenous CD33-positive cells such as endogenous CD33-positive malignant cells in the subject is performed after administering the genetically modified HSCs or HSPCs to the subject, administering.

Embodiment 40. The method of embodiment 39, further including the step of: after administering the genetically modified HSCs or HSPCs to the subject,
determining whether one or both of living endogenous CD33-positive hematological cells are present in the subject and living endogenous CD33-positive malignant hematological cells are present in the subject and when the determination of such cells being present in the subject is made, then performing the step of administering to the subject an amount of a radiolabeled anti-CD33 antibody effective to kill or inhibit the proliferation of the endogenous CD33-positive cells such as endogenous CD33-positive malignant hematological cells.

The determination of whether endogenous CD33-positive cells are present in the subject after hematopoietic recovery from the HSC or HSPC administration may, for example, be made from a biological sample obtained from the subject, such as a preobtained blood specimen or bone marrow specimen, by ELISA or immunohistochemistry using CD33-binding primary antibodies or by quantitative fluorescence flow cytometry using a CD33-binding antibody that is fluorescently labeled (or used with fluorescently labeled secondary antibodies) as known in the art, or by directly imaging CD33 expressing cells in the subject, for example, by PET imaging using a ⁸⁹Zr-labeled anti-CD33 antibody such as ⁸⁹Zr-labeled lintuzumab, such as ⁸⁹Zr-DFO-lintuzumab.

In embodiments of the invention in which the anti-CD33 and/or anti-CD45 antibody is labeled with ¹⁷⁷Lu, the following administered doses may, for example be used. The amount of the ¹⁷⁷Lu-labeled anti-CD33 or anti-CD45 antibody administered may, for example, be below 450 pCi/kg, 400 pCi/kg, 350 pCi/kg, 300 pCi/kg, 250 pCi/kg, 200 pCi/kg, 150 pCi/kg, 100 pCi/kg, 90 pCi/kg, 80 pCi/kg, 70 pCi/kg, 60 pCi/kg, 50 pCi/kg, 40 pCi/kg, 30 pCi/kg, 20 pCi/kg, 10 pCi/kg, 5 pCi/kg, or 1 pCi/kg. The amount of the ¹⁷⁷Lu-labeled anti-CD33 or anti-CD45 antibody administered may, for example, be at least 1 pCi/kg, 2.5 pCi/kg, 5 pCi/kg, 10 pCi/kg, 20 pCi/kg, 30 pCi/kg, 40 pCi/kg, 50 pCi/kg, 60 pCi/kg, 70 pCi/kg, 80 pCi/kg, 90 pCi/kg, 100 pCi/kg, 150 pCi/kg, 200 pCi/kg, 250 pCi/kg, 300 pCi/kg, 350 pCi/kg, 400 pCi/kg or 450 pCi/kg. The amount of the ¹⁷⁷Lu-labeled anti-CD33 or anti-CD45 antibody administered may, for example, be a dose that includes any combination of aforementioned upper and lower limits, such as from at least 5 pCi/kg to below 50 pCi/kg, or from at least 50 pCi/kg to below 500 pCi/kg.

The amount of the ¹⁷⁷Lu-labeled anti-CD33 or anti-CD45 antibody administered may, for example, be below 20 mCi, such as below 15 mCi, 10 mCi, 9 mCi, 8 mCi, 7 mCi, 6 mCi, 5 mCi, 3 mCi, 2 mCi, 1 mCi, 800 pCi, 600 pCi, 400 pCi, 200 pCi, 100 pCi, or 50 pCi. The amount of the ¹⁷⁷Lu-labeled anti-CD33 or anti-CD45 antibody administered may, for example, be at least 10 pCi, such as at least 25 pCi, 50 pCi, 100 pCi, 200 pCi, 300 pCi, 400 pCi, 500 pCi, 600 pCi, 700 pCi, 800 pCi, 900 pCi, 1 mCi, 2 mCi, 3 mCi, 4 mCi, 5 mCi, 10 mCi, or 15 mCi. The amount of the ¹⁷⁷Lu-labeled anti-CD33 or anti-CD45 antibody administered may, for example, be a dose that includes any combination of the aforementioned upper and lower limits, such as from at least 10 pCi to below 20 mCi, or from at least 100 pCi to below 3 mCi, or from 3 mCi to below 20 mCi.

Throughout this application, various patents, patent applications and other publications are cited, each of which is hereby incorporated by reference in its entirety.

While various specific embodiments have been illustrated and described herein, it will be appreciated that various changes can be made without departing from the spirit and scope of the invention(s). Moreover, features described in connection with one aspect of the invention may be used in conjunction with other aspects of the invention, even if not explicitly exemplified in combination within.

1.-36. (canceled)

37. A method for treating a hematological malignancy comprising CD33-positive malignant cells in a mammalian subject, comprising the steps of:
administering to the subject hematopoietic stem cells (HSCs) or hematopoietic stem and progenitor cells (HSPCs), genetically modified to reduce or eliminate the expression of CD33 antigen by or on myeloid lineage cells that descend from the HSCs or HSPCs; and
before and/or after administering the genetically modified HSCs or HSPCs to the subject, administering to the subject an amount of a radiolabeled anti-CD33 antibody effective to kill or inhibit the proliferation of CD33-positive endogenous cells in the subject,
wherein the radiolabeled anti-CD33 antibody is labeled with a radionuclide.

38. The method of claim 37, wherein
the administered genetically modified HSCs or HDPCs engraft in the subject and give rise to myeloid lineage cells with reduced or eliminated cell surface CD33 expression; and
the administered radiolabeled anti-CD33 antibody kills or inhibits the proliferation of endogenous CD33-positive cells in the subject.

39. The method of claim 37, wherein the radiolabeled anti-CD33 antibody comprises radiolabeled lintuzumab (HuM195), radiolabeled gemtuzumab, radiolabeled vadastuximab, a radiolabeled antibody comprising the heavy chain complementarity determining regions (CDRs) and/or the light chain CDRs of any of the preceding antibodies, a radiolabeled antibody comprising the heavy chain variable region and/or the light chain variable region of any of the preceding antibodies or a radiolabeled antigen-binding fragment of any of the preceding antibodies.

40. The method of claim 37 wherein the radionuclide comprises 131I, 125I, 123I, 90Y, 177Lu, 186Re, 188Re, 89Sr, 153Sm, 32P, 225Ac, 213Bi, 213Po, 211At, 212Bi, 213Bi, 223Ra, 227Th, 149Tb, 137Cs, 212Pb, 103Pd or any combination thereof.

41. The method of claim 40, wherein the radiolabeled anti-CD33 antibody is chemically conjugated to a chelator comprising 1,4,5,10-tetraazacyclododecance-1,4,7,10-tetracetic acid (DOTA) or a derivative thereof, and is labeled with the radionuclide 225Ac or 177Lu by chelation of the radionuclide by the conjugated chelator.

42. The method of claim 37, wherein the radiolabeled anti-CD33 antibody is 225Ac-labeled and the effective amount of the 225Ac-labeled antibody comprises a dose of 0.1 to 10 μCi/kg subject body weight.

43. The method of claim 42, wherein the radiolabeled anti-CD33 antibody is 225Ac-labeled and the effective amount of the 225Ac-labeled antibody comprises a dose of 0.5 to 4 μCi/kg subject body weight.

44. The method of claim 37, wherein the step of administering the radiolabeled anti-CD33 antibody is performed after the step of administering the genetically modified HSCs or HSPCs.

45. The method of claim 37, wherein the step of administering the radiolabeled anti-CD33 antibody is performed before the step of administering the genetically modified HSCs or HSPCs.

46. The method of claim 37, further comprising, the step of:
before administering the HSCs or HSPCs genetically modified to reduce or eliminate the expression of CD33 antigen by or on myeloid lineage cells that descend from the hematopoietic stem cells,
administering an amount of a radiolabeled anti-CD45 antibody effective to facilitate engraftment of genetically modified hematopoietic stems cells pursuant to their administration,
wherein the radiolabeled anti-CD45 antibody is labeled with a radionuclide comprising 131I, 125I, 123I, 90Y, ¹⁷⁷Lu, 186Re, 188Re, 89Sr, 153Sm, 32P, 225Ac, 213Bi, 213Po, 211At, 212Bi, 213Bi, 223Ra, 227Th, 149Tb, 137Cs, 212Pb, 103Pd or any combination thereof.

47. The method of claim 46, wherein the radiolabeled anti-CD45 antibody is labeled with 131I, 212Pb, 177Lu, or 225Ac.

48. The method of claim 47, wherein radiolabeled anti-CD45 antibody is labeled with ¹³¹I, and the amount administered is selected from the group consisting of from 10 mCi to 200 mCi, from 200 mCi to 400 mCi, and from 400 mCi to 1,200 mCi.

49. The method of claim 47, wherein the radiolabeled anti-CD45 antibody is labeled with 225Ac, the amount administered is selected from the group consisting of from 0.1 μCi/kg to 5.0 μCi/kg subject weight, from 0.1 μCi/kg to 1.0 μCi/kg subject weight, from 1.0 μCi/kg to 3.0 μCi/kg subject weight, and from 3.0 μCi/kg to 5.0 μCi/kg subject weight.

50. The method of claim 47, wherein the radiolabeled anti-CD45 antibody is labeled with 131I, and administered in an amount selected from the group consisting of: from 10 mCi to 200 mCi administered 6, 7, or 8 days before administering the genetically modified HSCs or HSPCs to the subject; from 200 mCi to 400 mCi administered 8, 9, 10, 11, or 12 days before administering the genetically modified HSCs or HSPCs to the subject; and from 400 mCi to 1,200 mCi of administered 10, 11, 12, 13, or 14 days before administering the genetically modified hematopoietic stem cells to the subject.

51. The method of claim 47, wherein the radiolabeled anti-CD45 antibody is labeled with ²²⁵Ac, and administered in an amount from 0.1 μCi/kg to 5.0 μCi/kg subject weight administered 6, 7, 8, 9, 10, 11, or 12 days before administering the genetically modified HSCs or HSPCs to the subject.

52. The method of claim 46, wherein the radiolabeled anti-CD45 antibody comprises:
immunoglobulin light chain CDRs having the amino acid sequences RASKSVSTSGYSYLH (LC-CDR-1; SEQ ID NO:3), LASNLES (LC-CDR-2; SEQ ID NO:4), and QHSRELPFT (LC-CDR-3; SEQ ID NO: 5); and/or
immunoglobulin heavy chain CDRs having the amino acid sequences GFDFSRYWMS (HC-CDR-1; SEQ ID NO:6), EINPTSSTINFTPSLKD (HC-CDR-2; SEQ ID NO:7) and GNYYRYGDAMDY (HC-CDR-3; SEQ ID NO:8).

53. The method of claim 52, wherein the radiolabeled antibody is radiolabeled apamistamab or an antigen-binding fragment thereof.

54. The method of claim 52, wherein the step of administering to the subject an amount of a radiolabeled anti-CD45 antibody comprising administering to the subject a composition comprising a radiolabeled fraction of the anti-CD33 antibody and a non-radiolabeled fraction of the anti-CD33 antibody in a ratio of 0.1:1 to 1:1 radiolabeled to non-radiolabeled.

55. The method of claim 54, wherein the amount of the anti-CD45 antibody in the composition administered is less than 16 mg/kg of subject body weight.

56. The method of claim 37, wherein the step of administering to the subject an amount of a radiolabeled anti-CD33 antibody comprises administering to the subject a composition comprising a radiolabeled fraction of the anti-CD33 antibody and a non-radiolabeled fraction of the anti-CD33 antibody in a ratio of 0.1:1 to 1:1 radiolabeled to non-radiolabeled.

57. The method of claim 56, wherein the amount of the anti-CD33 antibody in the composition administered is less than 16 mg/kg of subject body weight.

58. The method of claim 37, wherein the hematological malignancy is multiple myeloma, acute myeloid leukemia (AML), chronic myelogenous leukemia (CML), myelodysplastic syndrome (MDS), or a myeloproliferative neoplasm.

59. The method of claim 58, wherein the hematological malignancy is a relapsed and/or refractory.

60. The method of claim 37, wherein no conditioning agent other than a radiolabeled anti-CD45 antibody is administered to the subject prior to administration of the genetically modified HSCs or HSPCs to the subject.

61. The method of claim 37, wherein no external radiation is administered to the subject prior to and in preparation of the administration of the genetically modified HSCs or HSPCs to the subject.


SEQUENCE LISTING (22 sequences)

SEQ ID NO: 1 - protein, 111 aa, Mus musculus
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIK

SEQ ID NO: 2 - protein, 123 aa, Mus musculus
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAK

SEQ ID NO: 3 - protein, 15 aa, Mus musculus
RASKSVSTSGYSYLH

SEQ ID NO: 4 - protein, 7 aa, Mus musculus
LASNLES

SEQ ID NO: 5 - protein, 9 aa, Mus musculus
QHSRELPFT

SEQ ID NO: 6 - protein, 10 aa, Mus musculus
GFDFSRYWMS

SEQ ID NO: 7 - protein, 17 aa, Mus musculus
EINPTSSTINFTPSLKD

SEQ ID NO: 8 - protein, 12 aa, Mus musculus
GNYYRYGDAMDY

SEQ ID NO: 9 - DNA, 717 bp, Mus musculus
  source 1..717: mol_type = other DNA
atggagacagacacactcctgttatgggtactgctgctctgggttccaggttccactggtgacattgcgctgacacagtctcctgcttccttagctgtatctctgggacagagggccaccatctcatgcagggccagcaaaagtgtcagtacatctggctatagttatctgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 10 - DNA, 1389 bp, Mus musculus
  source 1..1389: mol_type = other DNA
atggattttgggctgattttttttattgttgctcttttaaaaggggtccagtgtgaggtgaagcttctcgagtctggaggtggcctggtgcagcctggaggatccctgaaactctcctgtgcagcctcaggattcgatttcagtagatactggatgagttgggtccggcaggctccagggaaagggctagaatggattggagagattaatccaactagcagtacgataaactttacgccatctctaaaggataaagtcttcatctccagagacaacgccaaaaatacgctgtacctgcaaatgagcaaagtgagatctgaggacacagccctttattactgtgcaagagggaactactataggtacggagatgctatggactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 11 - protein, 7 aa, Mus musculus
DIALTQS

SEQ ID NO: 12 - protein, 7 aa, Mus musculus
EVKLLES

SEQ ID NO: 13 - protein, 238 aa, Mus musculus
METDTLLLWVLLLWVPGSTGDIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 463 aa, Mus musculus
MDFGLIFFIVALLKGVQCEVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTDSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 463 aa, Mus musculus
MDFGLIFFIVALLKGVQCEVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 16 - protein, 211 aa, Mus musculus
PASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - protein, 438 aa, Mus musculus
GGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTDSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 18 - protein, 438 aa, Mus musculus
GGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 19 - DNA, 1952 bp, synthetic construct
  misc_feature 1..1952: note = coding sequence lintuzumab light chain
  source 1..1952: mol_type = other DNA
tctagaccaccatggagaaagacacactcctgctatgggtcctacttctctgggttccaggttccacaggtgacattcagatgacccagtctccgagctctctgtccgcatcagtaggagacagggtcaccatcacatgcagagccagcgaaagtgtcgacaattatggcattagctttatgaactggttccaacagaaacccgggaaggctcctaagcttctgatttacgctgcatccaaccaaggctccggggtaccctctcgcttctcaggcagtggatctgggacagacttcactctcaccatttcatctctgcagcctgatgacttcgcaacctattactgtcagcaaagtaaggaggttccgtggacgttcggtcaagggaccaaggtggagatcaaacgtaagtagaatccaaagtctagaaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgctctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctaggatcc

SEQ ID NO: 20 - protein, 238 aa, synthetic construct
  REGION 1..238: note = lintuzumab light chain
MEKDTLLLWVLLLWVPGSTGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 3246 bp, synthetic construct
  misc_feature 1..3246: note = coding sequence lintuzumab heavy chain
  source 1..3246: mol_type = other DNA
tctagaccaccatgggatggagctggatctttctcttcctcctgtcaggaactgctggcgtccactctcaggttcagctggtgcagtctggagctgaggtgaagaagcctgggagctcagtgaaggtttcctgcaaagcttctggctacaccttcactgactacaacatgcactgggtgaggcaggctcctggccaaggcctggaatggattggatatatttatccttacaatggtggtaccggctacaaccagaagttcaagagcaaggccacaattacagcagacgagagtactaacacagcctacatggaactctccagcctgaggtctgaggacactgcagtctattactgcgcaagagggcgccccgctatggactactggggccaagggactctggtcactgtctcttcaggtaagaatggcctctagaccaccatgggatggagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcacctgccctgggctgggccacgggaagccctaggagcccctggggacagacacacagcccctgcctctgtaggagactgtcctgttctgtgagcgccctgtcctccgacctccatgcccactcgggggcatgcctagtccatgtgcgtagggacaggccctccctcacccatctacccccacggcactaacccctggctgccctgcccagcctcgcacccgcatggggacacaaccgactccggggacatgcactctcgggccctgtggagggactggtgcagatgcccacacacacactcagcccagacccgttcaacaaaccccgcactgaggttggccggccacacggccaccacacacacacgtgcacgcctcacacacggagcctcacccgggcgaactgcacagcacccagaccagagcaaggtcctcgcacacgtgaacactcctcggacacaggcccccacgagccccacgcggcacctcaaggcccacgagcctctcggcagcttctccacatgctgacctgctcagacaaacccagccctcctctcacaagggtgcccctgcagccgccacacacacacaggggatcacacaccacgtcacgtccctggccctggcccacttcccagtgccgcccttccctgcaggatcc

SEQ ID NO: 22 - protein, 465 aa, synthetic construct
  REGION 1..465: note = lintuzumab heavy chain
MGWSWIFLFLLSGTAGVHSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK